(19) United States
(12) Patent Application Publication
(10) Pub. No.: US 2004/0071759 A1
(43) Pub. Date: Apr. 15, 2004

(54) Method and apparatus for decreasing the drowsiness of an individual

(76) Inventors: Michael R. Duff, Nashville, IN (US); Bradford G. Addison, Indianapolis, IN (US)

Correspondence Address: BARNES & THORNBURG, 11 SOUTH MERIDIAN, INDIANAPOLIS, IN, 46204

(21) Appl. No.: 10/645,844

(22) Filed: Aug. 22, 2003

Related U.S. Application Data
(63) Continuation of application No. 09/256,519, filed on Feb. 23, 1999, now abandoned.
(60) Provisional application No. 60/083,284, filed on Apr. 28, 1998.

(51) Int. Cl. A61K 9/70

(52) U.S. Cl. 424/445; 424/443

(57) ABSTRACT

A method of decreasing the drowsiness of an individual is disclosed. The method includes the steps of (i) removing a towelette from a dispenser, the towelette being impregnated with a stimulating organic substance and/or an ammonia containing substance, and (ii) contacting the skin of the individual with the towelette so that an amount of the stimulating organic substance and/or ammonia containing substance effective to decrease the drowsiness of the individual is transferred from the towelette to the skin of the individual. An associated apparatus is also disclosed.

### REFERENCE TO PRIOR APPLICATIONS

This application claims the benefit of U.S. Provisional Application Serial No. 60/083,284, filed Apr. 28, 1998 and U.S. application Ser. No. 09/256,519, filed Feb. 23, 1999, the disclosures of which are hereby incorporated by reference.

### BACKGROUND OF THE INVENTION

The present invention relates generally to a method and apparatus for decreasing the drowsiness of an individual, and more particularly, to a towelette impregnated with a substance for decreasing the drowsiness of an individual.

Some activities can periodically require an individual to be awake for extended periods of time. For example, sometimes truck drivers are required to drive for significant periods of time in order to deliver their cargo in a timely manner. In addition, college students are sometimes required to study late at night in order to properly prepare themselves for an examination. Individuals participating in these types of activities will often self administer various drugs to help them counteract the drowsiness they experience, and thus remain awake so as to complete the task.

The drugs self administered for the purpose of counteracting drowsiness typically include caffeine or an amphetamine. These drugs appear to be effective in counteracting drowsiness, however they also cause undesirable side effects. Specifically, the self administration of caffeine can cause sleep problems, restlessness, depression, stomach pains, rapid heart beat, headaches, and anxiety. Moreover, the chronic use of caffeine can result in the individual becoming addicted. As a result, the cessation of caffeine intake can precipitate a withdraw syndrome in the individual. The symptoms of caffeine withdraw can include feelings of fatigue, sedation, headaches, and nausea.

The undesirable side effects of amphetamines include restlessness, dizziness, tremor, hyperactive reflexes, tenseness, irritability, and weakness. As with caffeine, the chronic use of amphetamines can also result in the individual becoming addicted. The symptoms of amphetamine withdraw can include dysphoria, depression, sleepiness, fatigue, and bradycardia.

In addition to the above discussed undesirable side effects, caffeine and amphetamines also suffer from the drawback that it takes a relatively long time between the ingestion of these drugs until their antihypnotic effects are manifested. This is a disadvantage since individuals typically want an immediate decrease in drowsiness in the aforementioned situations.

What is needed therefore is a method and apparatus for decreasing the drowsiness of an individual which overcomes one or more of the above discussed drawbacks.

### SUMMARY OF THE INVENTION

In accordance with one embodiment of the present invention, there is provided a method of decreasing the drowsiness of an individual. The method includes the steps of (i) removing a towelette from a dispenser, the towelette being impregnated with a stimulating organic substance, and (ii) contacting the skin of the individual with the towelette so that an amount of the stimulating organic substance effective to decrease the drowsiness of the individual is transferred from the towelette to the skin of the individual.

Pursuant to another embodiment of the invention, there is provided a method of decreasing the drowsiness of an individual. The method includes the steps of (i) removing a towelette from a dispenser, the towelette being impregnated with an ammonia containing substance, and (ii) contacting the skin of the individual with the towelette so that an amount of the ammonia containing substance effective to decrease the drowsiness of the individual is transferred from the towelette to the skin of the individual.

In accordance with yet another embodiment of the invention, there is provided an apparatus for contacting the skin of an individual so as to decrease the drowsiness of the individual. The apparatus includes a towelette impregnated with a stimulating organic substance, wherein the stimulating organic substance is present on the towelette in an amount such that a quantity of the stimulating organic substance effective to decrease the drowsiness of the individual is transferred from the towelette to the skin of the individual when the towelette is placed in contact with the skin of the individual.

It is therefore an object of the present invention to provide a new and useful method and apparatus for decreasing the drowsiness of an individual.

It is also an object of present invention to provide an improved method and apparatus for decreasing the drowsiness of an individual.

It is yet another object of present invention to provide a safe and effective method and apparatus of decreasing the drowsiness of an individual.

It is also an object of present invention to provide a method and apparatus which rapidly decreases the drowsiness of an individual.

The above and other objects, features, and advantages of the present invention will become apparent from the following description and the attached drawings.

### BRIEF DESCRIPTION OF THE DRAWINGS

FIG. 1 is a perspective view of a towelette which incorporates features of the present invention therein, note that only a portion of the towelette is schematically shown impregnated with the substances of the present invention for clarity of description;

FIG. 2 is a fragmentary perspective view of a dispenser which contains a plurality of the towelettes of FIG. 1;

FIG. 3 is a top elevational view of another dispenser which contains a single towelette of FIG. 1;

FIG. 4 is a fragmentary perspective view of another dispenser which contains a plurality of the towelettes of FIG. 1; and

FIG. 5 is a front elevational view of a human head with the facial area thereof being contacted with a towelette of FIG. 1, note that the towelette is being grasped by a human hand and an amount of a substance of the present invention is schematically shown being transferred from the towelette to the skin of the facial area for clarity of description.

### DETAILED DESCRIPTION OF THE INVENTION

While the invention is susceptible to various modifications and alternative forms, a specific embodiment thereof has been demonstrated by way of example in the drawings and will herein be described in detail. It should be understood that there is no intent to limit the invention to the particular form disclosed, but on the contrary, the intention is to cover all modifications, equivalents, and alternatives falling within the spirit and scope of the invention as defined by the appended claims.

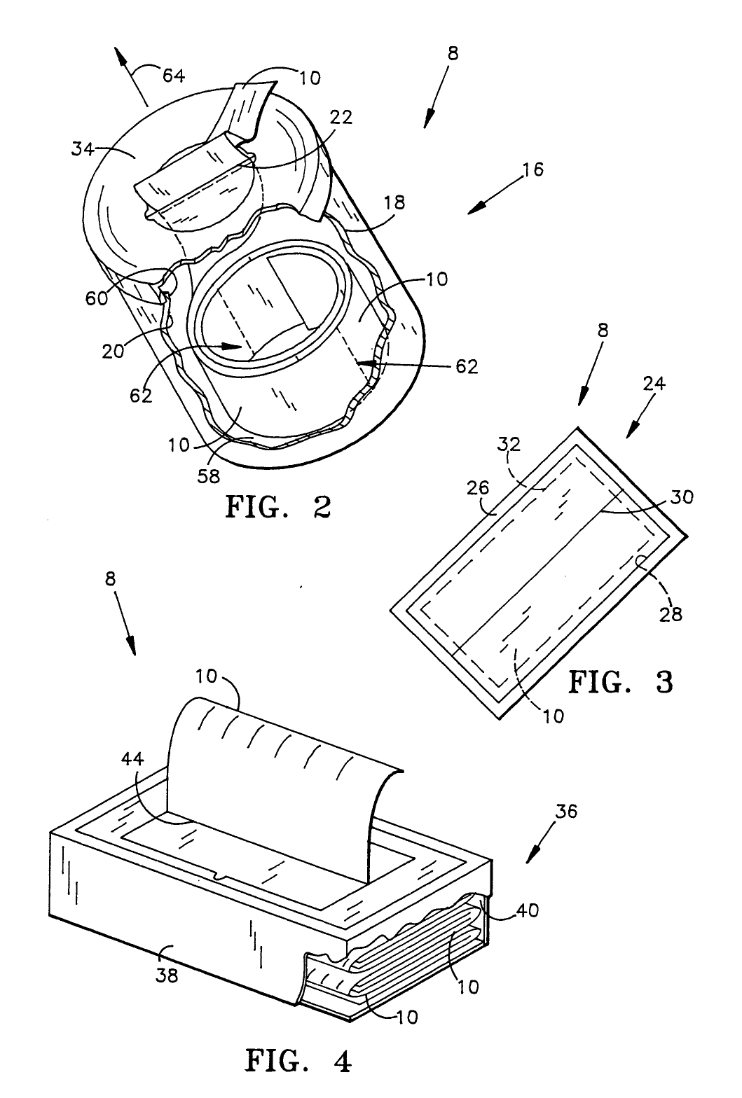

Referring now to FIGS. 2-4, there is shown three different embodiments of an apparatus 8 which incorporates the features of the present invention therein. Each embodiment of apparatus 8 includes a dispenser 16, 24, or 36 and a number of towelettes 10. In particular, the embodiment shown in FIG. 2 includes dispenser 16 which has a cylindrically shaped casing 18 and a cap 34. Casing 18 defines a cylindrically shaped cavity 20. Casing 18 includes a floor 58 and an open end 60. Casing 18 and cap 34 are preferably made of plastic, but may alternatively be made of any other material which prevents the evaporation of moisture contained in cavity 20. Cap 34 is constructed such that cap 34 can be secured to end 60 of casing 18. In addition, cap 34 has a slot 22 defined therein.

A plurality of towelettes 10 are disposed in a spiral orientation within cavity 20. It should be appreciated that towelettes 10 may comprise individual towelettes 10 or a single sheet of towelettes 10 having perforations 62 defined therein at intervals along the length thereof as shown in FIG. 2. The plurality of towelettes 10 are positioned within cavity 20 such that one towelette 10 extends through slot 22. Arranging towelettes 10 in the above described manner allows one towelette 10 to be removed from dispenser 16 at a time. Specifically, the towelette 10 extending out of slot 22 is pulled in a direction indicated by arrow 64 such that an end of the next towelette 10 also extends out of slot 22. Once the end of the next towelette 10 extends out of slot 22, the two towelettes 10 are torn apart along the perforation 62 interposed therebetween. The above described procedure is repeated when another towelette 10 is desired to be removed from dispenser 16. It should be appreciated that a secondary cap (not shown) for placing over slot 22 is contemplated. The secondary cap functions to prevent the end of the towelette 10 extending out of slot 22 from drying out before use.

The embodiment shown in FIG. 4 includes dispenser 36 which has a rectangularly shaped casing 38 having a slot 44 defined therein. Casing 38 defines a rectangularly shaped cavity 40. As with casing 18, casing 38 is preferably made of plastic, but may alternatively be made of any other material which prevents the evaporation of moisture contained in cavity 40.

A plurality of towelettes 10 are disposed in an accordian-fold orientation within a cavity 40 defined by casing 38 such that one towelette 10 extends out of slot 44. It should be appreciated that individual towelettes 10 can be removed from dispenser 36 much in the same way tissues are removed from a tissue dispenser. Moreover, it should be appreciated that a lid (not shown) for placing over slot 44 is contemplated. In a manner similar to that described above for the secondary cap, the lid functions to prevent the end of the towelette 10 extending out of slot 44 from drying out before use.

The embodiment shown in FIG. 3 includes dispenser 24 which has a casing 26 having a slot 30 defined therein. Casing 26 defines a cavity 28. A single towelette 10 is disposed within cavity 28. Dispenser 24 preferably includes a foil or plastic lined envelope 32 wherein the single towelette 10 is disposed within envelope 32 prior to placing into cavity 28 to prevent towelette 10 from drying out before use. Prior to use towelette 10 is removed from dispenser 24 by removing envelope 32 via slot 30 and then opening envelope 32 to gain access to towelette 10.

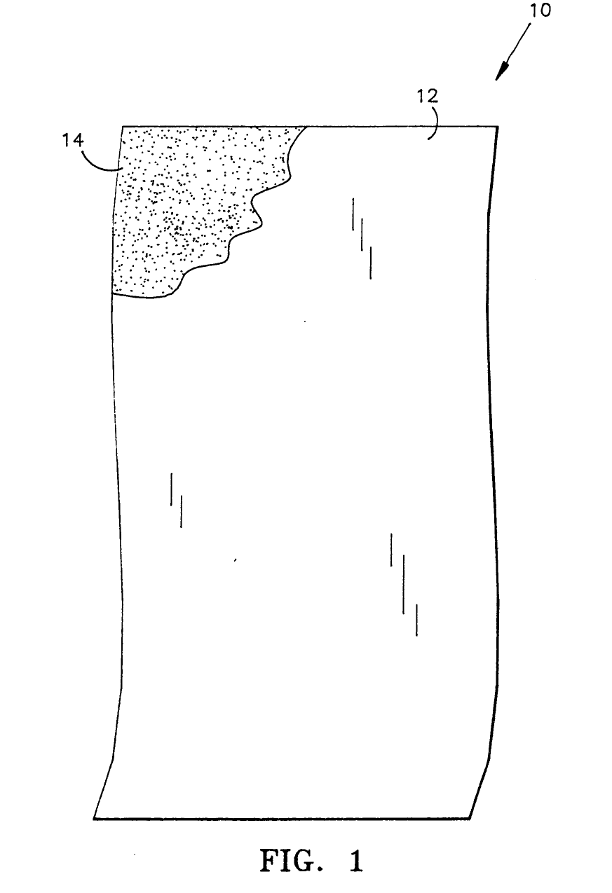

Referring now to FIG. 1, there is shown a towelette 10 which incorporates features of the present invention. Towelette 10 has a surface 12 that has a stimulating organic substance 14 or an ammonia containing substance 14 disposed thereon. Note that the stimulating organic substance and the ammonia containing substance are both indicated by the number 14 in FIG. 1 for clarity of description. However, it should be understood that towelette 10 can have (i) just a stimulating organic substance disposed thereon, (ii) just an ammonia containing substance disposed thereon, or (iii) a stimulating organic substance and an ammonia containing substance disposed thereon. Also note that only a portion of surface 12 is shown impregnated with organic substance 14 or ammonia containing substance 14 for clarity of description.

It should be understood that towelette 10 is formed of a liquid absorbent material which can be moistened and folded without structural deterioration. Towelette 10 should also have adequate strength when moistened to serve as a towel and yet be sufficiently soft to prevent any harm to the skin of the user during use. For example, towelette 10 can be made from absorbent paper or other nonwoven fabrics.

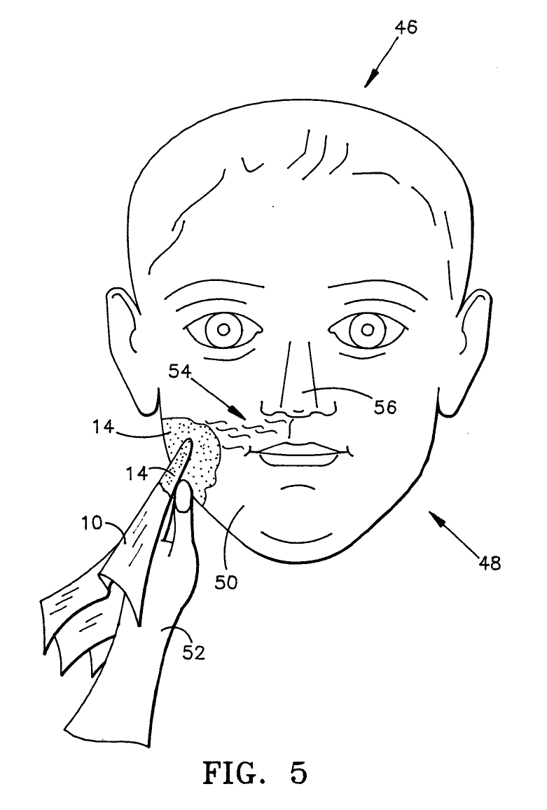

What is meant herein by a stimulating organic substance 14 is a chemical substance which is based on carbon chains or rings and also containing hydrogen with or without oxygen, nitrogen, or other elements. In addition, what is meant herein by stimulating organic substance 14 is a chemical substance which when placed in contact with human skin in an effective amount causes a decrease in the drowsiness of an individual 46 (see FIG. 5). What is meant herein by an ammonia containing substance is a chemical substance which contains ammonia (i.e. NH₃) or the ammonium ion (i.e. NH₄). What is also meant by an ammonia containing substance is a chemical substance which when placed in contact with human skin in an effective amount causes a decrease in the drowsiness of individual 46. What is meant herein by drowsiness is a decreased level of consciousness characterized by sleepiness and difficulty in remaining alert.

Preferably, stimulating organic substance 14 contains a chemical composition that when placed in contact with human skin in an effective amount causes (i) a stimulation of the nerve endings of the human body responsive for the detection of hot or cold so as to provide a sensation, such as a “cooling” sensation, on the skin of the individual 46 and (ii) a decrease in the drowsiness of the individual 46. It is preferable that the sensation (e.g. a topical “cooling” sensation) caused by stimulating organic substance 14 is a result of a direct stimulas on the nerve endings of the human body responsive for the detection of hot or cold, and not due to latent heat of evaporation. However, it should be understood that stimulating organic substances 14 which cause a sensation as a result of other sensory mechanisms, for example a topical cooling sensation caused by the heat evaporation of a stimulating organic substance 14 or an olfactory sensation caused by the individual breathing in vapors of a stimulating organic substance 14, can be utilized in the present invention as long as the sensation causes a decrease in the drowsiness of the individual.

Possible stimulating organic substances 14 which can be used in the present invention include menthol (i.e. (1α, 2β, 5α)-5-Methyl-2-(1-methylethyl)-cyclohexanol) which is commercially available from the Aldrich Corporation located in Milwaukee, Wis., as product number M2772; camphor (i.e. 1,7,7-Trimethylbicyclo[2.2.1]heptan-2-one) which is commercially available from the Aldrich Corporation as product number C352; methyl salicylate (i.e. 2-Hydroxybenzoic acid methyl ester) which is commercially available from the Aldrich Corporation as product number M80504; and eucalyptol (i.e. 1,3,3-Trimethyl-2-oxabicyclo[2.2.2]-octane) which is commercially available from the Aldrich Corporation as product number C80601.

Additional stimulating organic substances 14 which can be used in the present invention are ketals which are described in U.S. Pat. No. 5,451,404, Sep. 19, 1995 to Furman which is incorporated herein by reference. These ketals have the general formula

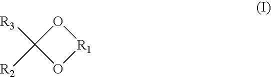

in which R₁ represents a C₂-C₆-alkylene radical having at least 1, but not more than 3, hydroxyl group(s), preferably 1 hydroxyl group, and either R₂ and R₃ independently of one another represent C₁-C₁₀-alkyl which is optionally substituted by 1 to 3 radicals selected from the group comprising hydroxyl, amino and halogen (such as fluorine, chlorine, bromine or iodine), C₅-C₇-cycloalkyl, preferably cyclohexyl, C₆-C₁₂-aryl, preferably phenyl, with the proviso that the total of the C atoms of R₂ and R₃ is not less than 3, or R₂ and R₃ together represent an alkylene radical which, together with the carbon atom which carries the radicals R₂ and R₃, forms a 5-7-membered ring, it being possible for this alkylene radical, in turn, to be substituted by C₁-C₆-aklyl groups.

Preferred radicals R₂ and R₃ comprise methyl, isopropyl and tert-butyl.

The length of the radicals R₂ and R₃ influences the effect of the compounds I: shorter radicals lead to an immediate, short effect; longer radicals lead to a delayed, but prolonged effect.

Preferred radicals R₁ embrace 1,2- and 1,3-alkylene radicals which, together with the two oxygen atoms and with the carbon atom to which the two oxygen atoms are attached, form a dioxolane or dioxane ring.

Preferred compounds I in which R₂ and R₃ together represent an alkylene radical are those of the general formula

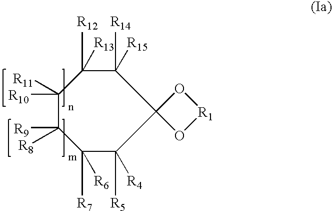

in which R₄ to R₁₅ independently of one another denote hydrogen or C₁-C₆-alkyl, preferably hydrogen or C₁-C₄-alkyl, and m and n independently of one another denote zero or 1.

Preferred compounds of the formula Ia are those in which the total of m+n is 1, i.e. ketals of an optionally substituted cyclohexanone.

Preferred substituents, of which there may be present, in particular, 1 to 3, are methyl, isopropyl and tert-butyl.

The ketals I can be prepared by known processes. For example, ketal I will generally be prepared by acid-catalysed reaction of the ketone on which ketal I is based and not less than the equivalent amount of aliphatic C₃-C₆-alcohol having not less than 3 and not more than 5, preferably 3, hydroxyl groups. In general, the ketone on which ketal I is based and not less than 0.5 equivalents, but, as a rule, a 1.2- to 4-fold, preferably 1.5- to 3-fold excess of this amount of the C₃-C₆-alcohol having 3 to 5 hydroxyl groups will be employed. Examples of acid catalysts which can be used are p-toluenesulphonic acid, phosphoric acid or potassium hydrogen sulphate in catalytically effective amounts (for example 0.1 to 3 g of p-toluenesuphonic acid per mole of ketone). The reaction will preferably be carried out either in an organic solvent which together with water forms an azeotrope, so that the water, which is liberated during the formation of the ketal, can be eliminated by azeotropic entrainment, or water-consuming coreagents such as, for example, trialkyl ortho esters are used. Examples of preferred organic solvents comprise benzene, toluene, xylene, chloroform, methylene chloride and trichloroethylene.

The reaction can be regarded as complete when water no longer separates out or when an ester/alcohol mixture is no longer separated out. It is recommended to wash the products subsequently with dilute alkali and with water, to separate and dry the organic phase, to strip off the solvent and, if appropriate, to purify the residue, for example by distillation.

Particularly preferred ketals I are those of the formulae

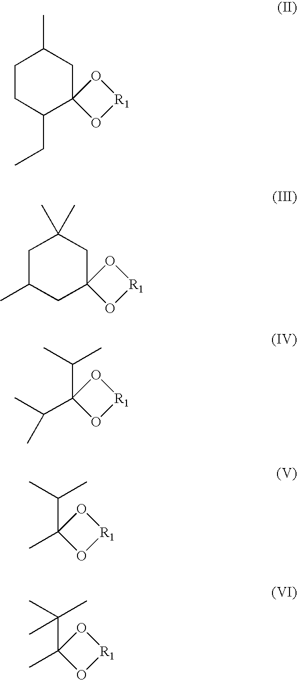

in which R₁ has the aforementioned meaning.

The ketals I to be employed in the compositions according to the invention can have asymmetric C atoms; optical isomerism can therefore occur. Depending on the starting material and the preparation methods used, they can exist in the form of mixtures of the optical isomers or in the form of pure isomers. The cooling effect of the isomers may differ, so that one or the other isomer may be preferred.

Moreover, various carboxamides can be utilized in the present invention as a stimulating organic substance 14. Several of these carboxamides are described in the aforementioned U.S. Pat. No. 5,451,404. In addition, carboxamides which can be utilized in the present invention as a stimulating organic substance 14 are described in U.S. Pat. No. 4,136,163, Jan. 23, 1979 to Wason et al., and U.S. Pat. No. 4,230,688, Oct. 28, 1980 to Rawsell et al. both of which are incorporated herein by reference.

Some of the aforementioned carboxamides are 3-substituted-p-menthanes of the general formula

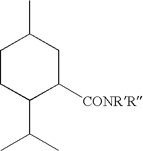

where R′, when taken separately, is hydrogen or an aliphatic radical containing up to 25 carbon atoms; R″ when taken separately is hydroxy, or an aliphatic radical containing up to 25 carbon atoms, with the proviso that when R′ is hydrogen R″ may also be an aryl radical of up to 10 carbon atoms and selected from the group consisting of substituted phenyl, phenalkyl or substituted phenalkyl, naphthyl and substituted naphthyl, pyridyl; and R′ and R″, when taken together with the nitrogen atom to which they are attached, represent a cyclic or heterocyclic group of up to 25 carbon atoms, e.g. piperidino, morpholino etc.

In the above definitions “aliphatic” is intended to include any straight-chained, branched-chained or cyclic radical free or aromatic unsaturation, and thus embraces alkyl, cycloalkyl, alkenyl, cycloalkenyl, alkynyl, hydroxyalkyl, acyloxyalkyl, alkoxy, alkoxyalkyl, aminoalkyl, acylaminoalkyl, carboxyalkyl and similar combinations.

Typical values for R′ and R″ when aliphatic are methyl, ethyl, propyl, butyl, isobutyl, n-decyl, cyclopropyl, cyclohexyl, cyclopentyl, cycloheptylmethyl, 2-hydroxyethyl, 3-hydroxy-n-propyl, 6-hydroxy-n-hexyl, 2-aminoethyl, 2-acetoxyethyl, 2-ethylcarboxyethyl, 4-hydroxybut-2-ynyl, carboxymethyl etc. When R″ is aryl typical values are benzyl, naphthyl, 4-methoxyphenyl, 4-hydroxyphenyl, 4-methylphenyl, 3-hydroxy-4-methylphenyl, 4-fluorophenyl, 4-nitrophenyl, 2-hydroxynaphthyl, pyridyl, etc.

The above described 3-substituted-p-menthanes may be readily prepared by conventional methods, such as by the reaction of the corresponding acid chloride (obtained by reacting p-menthane-3-carboxylic acid with thionyl chloride) with the appropriate mono or di-substituted amine. The reaction will usually be carried out in solution in the presence of a hydrogen chloride receptor, e.g. sodium hydroxide. The reaction proceeds smoothly at room temperature.

Furthermore some of the aforementioned carboxamides are certain acyclic tertiary and secondary carboxamides. These have the general formula

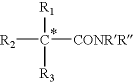

where R′ and R″, when taken separately, are each hydrogen, C₁-C₅ alkyl or C₁-C₈ hydroxyalkyl and provide a total of no more than 8 carbon atoms, with the proviso that when R′ is hydrogen R″ may also be alkylcarboxyalkyl of up to 6 carbon atoms; R′ and R″, when taken together, represent an alkylene group of up to 6 carbon atoms, the opposite ends of which group are attached to the amide nitrogen atom thereby to form a nitrogen heterocycle, the carbon chain of which may optionally be interrupted by oxygen;

R₁ is hydrogen or C₁-C₅ alkyl; and R₂ and R₃ are each C₁-C₅ alkyl; with the provisos that (i) R₁, R₂ and R₃ together provide a total of at least 5 carbon atoms, preferably from 5-10 carbon atoms; and (ii) when R₁ is hydrogen, R₂ is C₂-C₅ alkyl and R₃ is C₃-C₅ alkyl and at least one of R₂ and R₃ is branched, preferably in an alpha or beta position relative to the carbon atom marked (*) in the formula.

The above described carboxamides may readily be prepared by conventional techniques, for example, by reaction of an acid chloride of the formula R₁R₂R₃COCl with an amine of the formula HNR′R″ in the presence of a hydrogen chloride acceptor.

U.S. Pat. No. 5,725,865, Mar. 10, 1998 to Mane et al., which is incorporated herein by reference, also describes additional, succinate-based, stimulating organic substances 14 which can be used in the present invention. These stimulating organic substances 14 include monmenthyl succinate, monomenthyl sodium succinate, monomenthyl potassium succinate, monomenthyl lithium succinate, monomenthyl calcium succinate, monomenthyl magnesium succinate, monomenthyl barium succinate, alkali metal salts of monomenthyl succinate, and alkaline earth metal salts of monomenthyl succinate as well as mixtures thereof. The article “A Molecular Approach to Flavor Synthesis. I. Menthol Esters of Varying Size and Polarity.” Jabloner, H. and Dunbar, B. I., J. of Polymer science, Vol. 18, pages 2933-2940 (1980), which is incorporated herein by reference, discloses a method for the synthesis of monomenthyl succinate as well as monomenthyl sodium succinate and other menthol esters derived from monomenthyl succinate.

Possible ammonia containing substances 14 which can be used in the present invention include an aqueous solution of ammonia and an aqueous solution of ammonia carbonate. Ammonia carbonate is commercially available from the Sigma Corporation located in St. Louis, Mo., as product number A9516.

The stimulating organic substances 14 and the ammonia containing substances 14 used in the preparation of towelette 10 of the present invention may be carried in (e.g. dissolved in) a safe and effective amount of a topical pharmaceutically-acceptable carrier or diluent which can be of a variety of different forms. The following discussion is directed only to the stimulating organic substances 14, however it should be understood that the discussion also applies to any ammonia containing substance 14 which is used in the present invention.

By “safe and effective” is meant an amount of the pharmaceutically-acceptable carrier sufficient to act as a suitable vehicle for the stimulating organic substances 14, and any other optional components, but not so much as to cause any adverse side effects or skin reactions. In addition, “safe and effective” means the pharmaceutically-acceptable carrier contains a quantity of one or more stimulating organic substances 14 such that when towelette 10 contacts the skin 50 (see FIG. 5) of an individual 46 an amount of stimulating organic substance 14 effective to decrease the drowsiness of the individual 46 is transferred from towelette 10 to the skin 50 of the individual 46, but not so much as to cause any adverse side effects or skin reactions.

With respect to a composition of a pharmaceutically-acceptable carrier and one or more stimulating organic substances 14 not causing any adverse side effects or skin reactions, several patch test methods are available for determining the skin irritancy and sensitization caused by topically applied compositions. These test methods involve the use of occlusive dressings impregnated with the composition to be tested. These dressings are applied at various time intervals to selected sites on a test subject's skin such that the time intervals allow rest periods for possible sensitization to develop. Responses occurring within several days may indicate irritancy. These areas are then challenged with the test composition after the rest periods to determine whether sensitization has occurred. Examples of the above described patch test methods include (i) the Draize human skin irritancy and sensitization tests and its various modifications which is described in Draize, J. H., “Appraisal of Safety of Chemicals in Foods, Drugs and Cosmetics,” The Association of Food and Drug Officials of the United States, Texas State Department of Health, Austin, Tex., pp. 46-59, 1959, (ii) the method of Shelanski and Shelanaki as described in Shelanski, H. A. and M. V. Shelanski, “A New Technique of Human Patch Tests,” in “Proceedings of Scientific Section of the Toilet Goods Association,” 19:46-49,1953, and (iii) the maximization procedure of Kilgman or its modifications as described in Kilgman, A. M., “The Identification of Contact Allergens by Human Assay III. The Maximization Test: A procedure for Screening and Rating Contact Sensitizers,” Journal of Investigative Dermatology, 47:393-409, 1966 all of which are incorporated herein by reference.

With respect to the particular amount required of any stimulating organic substance 14 to effectively decrease the drowsiness of the individual 46 when transferred from towelette 10 to the skin 50 of the individual 46, this amount will vary depending upon the particular stimulating organic substance 14 used and the strength of the anti-drowsiness effect desired. However, various methods and apparatus are available for accessing the drowsiness or wakefulness of an individual which can be used to determine and optimize the amount of stimulating organic substance 14 required to effectively decrease the drowsiness of the individual 46 when the stimulating organic substance 14 is transferred from towelette 10 to the skin 50 of the individual 46. Thus, it should be understood that the data collected from the above described methods and apparatus in conjunction with the data collected with the aforementioned patch test methods can be used to determine and optimize the various physical and chemical characteristics of the present invention. For example, the above described methods and apparatus in conjunction with the aforementioned patch test methods can be used to determine and optimize (i) the concentration of the stimulating organic substance 14 in the pharmaceutically-acceptable carrier and (ii) the amount of the of the stimulating organic substance 14/pharmaceutically-acceptable carrier composition impregnated or disposed on towelette 10.

Furthermore, these drowsiness accessing methods and apparatus can be utilized to routinely determine whether a topically applied organic chemical compound or ammonia containing composition is capable of decreasing the drowsiness of an individual. Therefore, since a stimulating organic substance 14 and an ammonia containing substance 14 of the present invention must be capable of decreasing the drowsiness of an individual when topically applied, these methods and apparatus can also be utilized to routinely determine which organic chemical compounds or ammonia containing compositions can be used as a stimulating organic substance 14 and an ammonia containing substance 14, respectively, in the present invention.

Examples of the aforementioned drowsiness accessing methods and apparatus include (i) the Wilkison vigilance test as described in Prog. Clin. Psychol., 8, 28-43 (1968) and (ii) a method and apparatus for monitoring and estimating the awakeness of a person as described in U.S. Pat. No. 5,846,206, Dec. 8, 1998, to Bader, both of which are incorporated herein by reference. It should be understood that both of the above documents describe methods and apparatus which can be used to routinely determine and optimize the amount of stimulating organic substance 14 required to effectively decrease the drowsiness of the individual 46 when the stimulating organic substance 14 is transferred from towelette 10 to the skin 50 of the individual 46.

“Pharmaceutically-acceptable” means that the carrier is suitable for topical application to the skin without causing any untoward safety or toxicity concerns. In other words, these carriers are suitable for use on mammalian skin. For example, possible carriers include hydroalcoholic systems (e.g. liquids and gels), an anhydrous oil or silicone based system, or an emulsion system including, but not limited to, oil-in-water, water-in-oil, water-in-oil-in-water, and oil-in-water-in-silicone emulsions. The emulsions can cover a broad range of consistencies including thin lotions, creamy lotions, light creams, heavy creams, and the like. The emulsions can also include microemulsion systems. Other suitable topical carriers include anhydrous solids and semisolids (such as gels); and aqueous based mousse systems. Nonlimiting examples of the topical carrier systems useful in the present invention are described in the following four references, all of which are incorporated herein by reference: “Sun Products Formulary”, Cosmetics & Toiletries, vol. 105, pp. 122-139 (December 1990); “Sun Products Formulary”, Cosmetics & Toiletries, vol. 102, pp. 117-136 (March 1987); U.S. Pat. No. 4,960,764 to Figueroa et al., Oct. 2, 1990; and U.S. Pat. No. 4,254,105 to Fukuda et al., Mar. 3, 1981.

The pharmaceutically-acceptable topical carriers, in total, can comprise from about 0.1% to about 99% by weight of the compositions (i.e. the stimulating organic substance 14/pharmaceutically-acceptable carrier compositions and/or the ammonia containing substance 14/pharmaceutically-acceptable carrier compositions) useful in the present invention.

In particular, the above described ketals can be used with the pharmaceutically-acceptable topical carriers in the present invention in any safe and effective amount. For example, the ketals can generally make up (by weight) from about 0.0007% to about 0.6% of the stimulating organic substance 14/pharmaceutically-acceptable carrier composition.

The above described carboxamides can be used with the pharmaceutically-acceptable topical carriers in the present invention in any safe and effective amount. For example, the carboxamides can generally make up (by weight) from about 0.0005% to about 0.7% of the stimulating organic substance 14/pharmaceutically-acceptable carrier composition.

The above described succinate-based compounds can also be used with the pharmaceutically-acceptable topical carriers in the present invention in any safe and effective amount. For example, the succinate-based compounds can generally make up (by weight) from about 0.001% to about 1.0% of the stimulating organic substance 14/pharmaceutically-acceptable carrier composition.

Menthol, camphor, methyl salicylate, and eucalyptol can also be used with the pharmaceutically-acceptable topical carriers in the present invention in any safe and effective amount. For example, menthol can generally make up (by weight) from about 1.0% to about 3.0% of the stimulating organic substance 14/pharmaceutically-acceptable carrier composition. Camphor can generally make up (by weight) from about 0.01% to about 11.0% of the stimulating organic substance 14/pharmaceutically-acceptable carrier composition. Methyl salicylate can generally make up (by weight) from about 15.0% to about 30.0% of the stimulating organic substance 14/pharmaceutically-acceptable carrier composition. Eucalyptol can generally make up (by weight) from about 1.0% to about 3.0% of the stimulating organic substance 14/pharmaceutically-acceptable carrier composition.

The above described ammonia containing substances can be used with the pharmaceutically-acceptable topical carriers in the present invention in any safe and effective amount. In particular, an aqueous solution of ammonia can generally make up (by weight) from about 0.25% to about 5.0% of the ammonia containing substance 14/pharmaceutically-acceptable carrier mixture. Ammonia carbonate can generally make up (by weight) from about 0.25% to about 5.0% of the ammonia containing substance 14/pharmaceutically-acceptable carrier mixture.

A wide variety of acids, bases and buffers can be utilized to adjust and/or maintain the pH of the stimulating organic substance 14/pharmaceutically-acceptable carrier compositions useful in the present invention. These acids, bases and buffers can also be utilized to adjust and/or maintain the pH the ammonia containing substance 14/pharmaceutically-acceptable carrier compositions useful in the present invention. Examples of materials useful for adjusting and/or maintaining the pH include triethanolamine, sodium carbonate, sodium hydroxide, hydrochloric acid, phosphoric acid, sodium hydrogen phosphate, sodium dihydrogen phosphate, citric acid, and the like.

It should be understood that “masking” aromas can also be used in conjunction with the ammonia containing substance 14 and/or the stimulating organic substance 14. For example, substances that give off a lemon, mint, coffee, melon, or spice aroma can be used in the present invention to “mask” the smell of the ammonia containing substance 14.

As shown in FIG. 5, during use of apparatus 8 individual 46 removes a towelette 10 from a dispenser (e.g. dispenser 16; see FIG. 2) with their hand 52. The individual 46 then preferably contacts the skin 50 of the facial area 48 with the towelette 10 so that an amount of the stimulating organic substance 14, or the ammonia containing substance 14, effective to decrease the drowsiness of the individual 46 is transferred from the towelette 10 to the skin 50 of the individual 46. Preferably, once the above described amount of the stimulating organic substance 14, or the ammonia containing substance 14, is transferred to the skin 50 of the individual 46, vapors 54 of the stimulating organic substance 14, or the ammonia containing substance 14, are created (e.g. by evaporation). Once the aforementioned vapors 54 are created, the individual 46 can breathe in the vapors 54 (e.g. through the nose 56) such that the vapors 54 decrease the drowsiness of the individual 46, or enhance the anti-drowsiness effect of the stimulating organic substance 14 or the ammonia containing substance 14.

It should be understood that preferably towelette 10 of the present invention is used in the above described manner during the performance of a work function by the individual 46. For example, the towelette 10 can be used in a situation where the individual 46 is involved in driving a truck. Specifically, the towelette 10 is useful when the individual 46 is driving a truck for a first period of time under the influence of a first degree of drowsiness. Under this situation the individual 46 utilizes the towelette 10 as described above such that the drowsiness of the individual 46 is decreased to a second degree of drowsiness which is less that the first degree of drowsiness. Once the drowsiness of the individual 46 is decreased to the second degree of drowsiness, the individual 46 can continue with the performance of the work function in a more effective manner.

While the invention has been illustrated and described in detail in the drawings and foregoing description, such an illustration and description is to be considered as exemplary and not restrictive in character, it being understood that only the preferred embodiment has been shown and described and that all changes and modifications that come within the spirit of the invention are desired to be protected.

What is claimed is:

1. A method of decreasing the drowsiness of an individual, comprising the steps of:
removing a towelette from a dispenser, said towelette being impregnated with a stimulating organic substance; and
contacting skin of said individual with said towelette so that an amount of said stimulating organic substance effective to decrease said drowsiness of said individual is transferred from said towelette to said skin of said individual.

2. The method of claim 1, further comprising the steps of:
vaporizing a quantity of said stimulating organic substance transferred from said towelette to said skin of said individual so as to create vapors of said stimulating organic substance; and
said individual breathing in an amount of said vapors of said stimulating organic substance effective to decrease said drowsiness of said individual.

3. The method of claim 1, wherein:
said skin is located on a facial area of said individual.

4. The method of claim 1, wherein:
said dispenser includes a casing which defines a cavity, and
said cavity contains a number of said towelettes.

5. The method of claim 1, wherein:
said stimulating organic substance is dispersed in a pharmaceutically acceptable carrier.

6. The method of claim 5, wherein:
said stimulating organic substance includes a material selected from the group consisting of camphor, methyl salicylate, menthol, and eucalyptol.

7. The method of claim 6, wherein:
a mixture of said stimulating organic substance and said pharmaceutically acceptable carrier contains about 0.01% to about 11% camphor.

8. The method of claim 6, wherein:
a mixture of said stimulating organic substance and said pharmaceutically acceptable carrier contains about 15% to about 30% methyl salicylate.

9. The method of claim 6, wherein:
a mixture of said stimulating organic substance and said pharmaceutically acceptable carrier contains about 1.0% to about 3.0% menthol.

10. The method of claim 6, wherein:
a mixture of said stimulating organic substance and said pharmaceutically acceptable carrier contains about 1.0% to about 3.0% eucalyptol.

11. The method of claim 5, wherein:
said stimulating organic substance includes a material selected from the group consisting of monomenthyl succinate, alkali metal salts of monomenthyl succinate, alkaline earth metal salts of monomenthyl succinate, carboxamides, and ketals.

12. The method of claim 11, wherein:
said carboxamide is selected from the group consisting of, 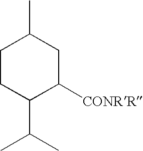
where R′, when taken separately, is hydrogen or an aliphatic radical containing up to 25 carbon atoms; R″ when taken separately is hydroxy, or an aliphatic radical containing up to 25 carbon atoms, with the proviso that when R′ is hydrogen R″ may also be an aryl radical of up to 10 carbon atoms and selected from the group consisting of phenyl, phenalkyl, naphthyl, and pyridyl; and R′ and R″, when take together with the nitrogen atom to which they are attached, represent a cyclic or heterocylic group of up to 25 carbon atoms; and
acyclic tertiary and secondary carboxamides of the formula: 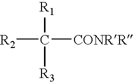
where R′ and R″, when taken separately, are each hydrogen, C₁-C₅ alkyl or C₁-C₈ hydroxylalkyl and provide a total of no more than 8 carbon atoms, with the proviso that when R′ is hydrogen R″ may also be alkylcarboxyalkyl of up to 6 carbon atoms; R′ and R″, when taken together, represent an alkylene group of up to 6 carbon atoms, the opposite ends of which group are attached to the amide nitrogen atom thereby to a form a nitrogen heterocycle, the carbon chain of which may optionally be interrupted by oxygen; R₁ is hydrogen or C₁-C₅ alkyl; and R₂ and R₃ are each C₁-C₅ alkyl; with the provisos that (i) R₁, R₂ and R₃ together provide a total of at least 5 carbon atoms; and (ii) when R₁ is hydrogen, R₂ is C₂-C₅ alkyl and R₃ is C₂-C₅ alkyl and at least one of R₂ and R₃ is branched and mixtures thereof.

13. The method of claim 11 wherein:
said ketal is selected from the group consisting of, 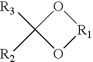
in which R₁ represents a C₂-C₆ alkylene radical having at least 1, but not more than 3, hydroxyl group(s), and either R₂ and R₃ independently of one another represent C₁-C₁₀-alkyl which is optionally substituted by 1 to 3 radicals selected from the group consisting of hydroxyl, amino, halogen, C₅-C₇-cycloalkyl, C₆-C₁₂-aryl, with the proviso that the total of the C atoms of R₂ and R₃ is not less than 3, or R₂ and R₃ together represent an alkylene radical which, together with the carbon atom which carries the radicals R₂ and R₃, forms a 5-7 membered ring, optionally substituted by C₁-C₆-alkyl groups.

14. A method of decreasing the drowsiness of an individual, comprising the steps of:
removing a towelette from a dispenser, said towelette being impregnated with an ammonia containing substance; and
contacting skin of said individual with said towelette so that an amount of said ammonia containing substance effective to decrease said drowsiness of said individual is transferred from said towelette to said skin of said individual.

15. The method of claim 14, further comprising the steps of:
vaporizing a quantity of ammonia from said ammonia containing substance transferred from said towelette to said skin of said individual so as to create ammonia vapors; and
said individual breathing in an amount of said ammonia vapors of said ammonia containing substance effective to decrease said drowsiness of said individual.

16. The method of claim 14, wherein:
said skin is located on a facial area of said individual.

17. The method of claim 14, wherein:
said dispenser includes a casing which defines a cavity, and
said cavity contains a number of said towelettes.

18. The method of claim 14, wherein:
said ammonia containing substance is dispersed in a pharmaceutically acceptable carrier.

19. The method of claim 18, wherein:
said ammonia containing substance is an aqueous solution of ammonia or ammonia carbonate.

20. The method of claim 19, wherein:
a mixture of said aqueous solution of ammonia and said pharmaceutically acceptable carrier contains about 0.25% to about 5.0% ammonia.

21. The method of claim 19, wherein:
a mixture of said ammonia carbonate and said pharmaceutically acceptable carrier contains about 0.25% to about 5.0% ammonia carbonate.

22. An apparatus for contacting the skin of an individual so as to decrease the drowsiness of said individual, comprising:
a towelette impregnated with a stimulating organic substance, wherein said stimulating organic substance is present on said towelette in an amount such that a quantity of said stimulating organic substance effective to decrease said drowsiness of said individual is transferred from said towelette to said skin of said individual when said towelette is placed in contact with said skin of said individual.

23. The apparatus of claim 22, further comprising:
a dispenser which includes a casing that defines a cavity, wherein said cavity contains a number of said towelettes.

24. The apparatus of claim 22, wherein:
said stimulating organic substance is dispersed in a pharmaceutically acceptable carrier.

25. The apparatus of claim 24, wherein:
said stimulating organic substance includes a material selected from the group consisting of camphor, methyl salicylate, and menthol.

26. The apparatus of claim 25, wherein:
a mixture of said stimulating organic substance and said pharmaceutically acceptable carrier contains about 0.01% to about 11% camphor.

27. The apparatus of claim 25, wherein:
a mixture of said stimulating organic substance and said pharmaceutically acceptable carrier contains about 15% to about 30% methyl salicylate.

28. The apparatus of claim 25, wherein:
a mixture of said stimulating organic substance and said pharmaceutically acceptable carrier contains about 1.0% to about 3.0% menthol.

29. The apparatus of claim 22, wherein:
said stimulating organic substance includes a material selected from the group consisting of monomenthyl succinate, alkali metal salts of monomenthyl succinate, alkaline earth metal salts of monomenthyl succinate, carboxamides, and ketals.

30. The apparatus of claim 29, wherein:
said carboxamide is selected from the group consisting of, 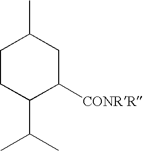
where R′, when taken separately, is hydrogen or an aliphatic radical containing up to 25 carbon atoms; R″ when taken separately is hydroxy, or an aliphatic radical containing up to 25 carbon atoms, with the proviso that when R′ is hydrogen R″ may also be an aryl radical of up to 10 carbon atoms and selected from the group consisting of phenyl, phenalkyl, naphthyl, and pyridyl; and R′ and, R″, when take together with the nitrogen atom to which they are attached, represent a cyclic or heterocylic group of up to 25 carbon atoms; and
acyclic tertiary and secondary carboxamides of the formula: 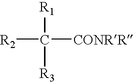
where R′ and R″, when taken separately, are each hydrogen, C₁-C₅ alkyl or C₁-C₈ hydroxylalkyl and provide a total of no more than 8 carbon atoms, with the proviso that when R′ is hydrogen R″ may also be alkylcarboxyalkyl of up to 6 carbon atoms; R′ and R″, when taken together, represent an alkylene group of up to 6 carbon atoms, the opposite ends of which group are attached to the amide nitrogen atom thereby to a form a nitrogen heterocycle, the carbon chain of which may optionally be interrupted by oxygen; R₁ is hydrogen or C₁-C₅ alkyl; and R₂ and R₃ are each C₁-C₅ alkyl; with the provisos that (i) R₁, R₂ and R₃ together provide a total of at least 5 carbon atoms; and (ii) when R₁ is hydrogen, R₂ is C₂-C₅ alkyl and R₃ is C₂-C₅ alkyl and at least one of R₂ and R₃ is branched and mixtures thereof.

31. The apparatus of claim 29 wherein:
said ketal is selected from the group consisting of, 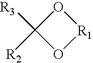
in which R₁ represents a C₂-C₆ alkylene radical having at least 1, but not more than 3, hydroxyl group(s), and either R₂ and R₃ independently of one another represent C₁-C₁₀-alkyl which is optionally substituted by 1 to 3 radicals selected from the group consisting of hydroxyl, amino, halogen, C₅-C₇-cycloalkyl, C₆-Cl₂-aryl, with the proviso that the total of the C atoms of R₂ and R₃ is not less than 3, or R₂ and R₃ together represent an alkylene radical which, together with the carbon atom which carries the radicals R₂ and R₃, forms a 5-7 membered ring, optionally substituted by C₁-C₆-alkyl groups.
